(19) United States
(12) Patent Application Publication
(10) Pub. No.: US 2012/0029294 A1
(43) Pub. Date: Feb. 2, 2012

(54) CANNULA

(76) Inventor: Eric Smith, Waban, MA (US)

(21) Appl. No.: 13/257,800

(22) PCT Filed: Apr. 1, 2010
(86) PCT No.: PCT/US10/29599

§ 371 Date: Oct. 18, 2011

Related U.S. Application Data
(60) Provisional application No. 61/166,971, filed on Apr. 6, 2009.

(51) Int. Cl. A61B 1/32

(52) U.S. Cl. 600/205

(57) ABSTRACT

A device for introducing surgical tools into a body is provided that a slotted cannula supported on a handle. The cannula is an elongate plate having a first end rigidly connected to an end of the handle, and a second end opposed to the first end. The plate is curved in the width direction so that the cannula has a cross-sectional shape that is a minor arc opening upward. As a result of this configuration, the cannula has a concave tool receiving surface, and an opposed, convex outer surface. A cannula fluid channel is disposed between the tool-receiving and outer surfaces. The cannula fluid channel extends from the handle to the second end of the cannula, and opens to the cannula exterior at the leading tip of the cannula.

### CROSS REFERENCE TO RELATED APPLICATIONS

This application claims the benefit of the priority U.S. Provisional Application 61/166,971, filed Apr. 6, 2009, the contents of which are incorporated herein by reference.

### BACKGROUND OF THE INVENTION

1. Field of the Invention

The present invention is related to surgical instruments and, more specifically is related to a cannula for use in arthroscopic procedures.

2. Description of the Background Art

It is well known to use specialized tools to aid insertion of surgical instruments such as arthroscopes, cutting and/or trimming tools, clamps, optical devices, etc. into a body for use during arthroscopic procedures in which movable joints are repaired, replaced or surgically constructed. For example, a cannula in the form of a rigid tube is often inserted into the body and used to direct the surgical instruments to a desired location in an operative field while minimizing trauma to surrounding tissue.

Conventional cylindrical cannulas are limited to use with instruments having an outer diameter less than that of the inner diameter of the cannula. Moreover, the leading tip of the cannula may be submerged in pooled body or irrigation fluids in the operative field whereby visualization of the joint, and position of the leading tip relative to the joint, is obscured. In these situations, accurate placement of the leading tip relative to the joint, and ultimately, placement of the inserted instrument, can be difficult.

### SUMMARY

A device is provided for introducing surgical tools into a body. The device includes a handle and a cannula extending from the handle. The cannula has a first end fixed to the handle, and a second end opposed to the first end which corresponds to the leading tip. The cannula has a cross sectional shape that is a minor arc which defines a concave tool-receiving surface, and a convex outer surface opposed to the tool-receiving surface. The tool-receiving and outer surfaces extend axially from the handle to the second end of the cannula. In addition, a cannula fluid channel is disposed between the tool-receiving and outer surfaces. The cannula fluid channel extends from the handle to the second end of the cannula, and opens to the cannula exterior at the second end (leading tip) of the cannula.

Advantageously, since the peripheral shape of cannula has the form of a minor arc, the device provides an arcuately shaped, trough-like surface which receives and guides surgical instruments, and is not limited to instruments of any particular diameter. Also advantageously, by providing a fluid channel within the body of the cannula, waste irrigation fluid, blood, or tissue fragments can be removed from the vicinity of the leading tip of the cannula by drawing them into the fluid channel so as to permit clear visualization of the joint and the position of the device relative to the joint. Alternatively, the fluid channel can also be used to introduce irrigation fluids into the surgical field.

In some aspects, a device for introducing surgical tools into a body is provided. The device includes a handle, and a cannula extending from the handle. The cannula includes a first end fixed to the handle, and has a shape that is a minor arc in cross section. The arcuately shaped cannula defines a concave tool-receiving surface, and a convex outer surface opposed to the tool-receiving surface. The tool-receiving and outer surfaces extend axially from the handle to a second end of the cannula that is opposed to the first end, and a cannula fluid channel is disposed between the tool-receiving and outer surfaces. The cannula fluid channel extends from the handle to the second end of the cannula, and opens to the cannula exterior at the second end of the cannula.

The device may include one or more of the following features: The minor arc has a circumferential arc length in a range between 20 degrees and 180 degrees. The cannula fluid channel is arcuate in cross sectional shape. The cannula further includes opposed sidewalls that join peripheral side edges of the tool-receiving surface and the outer surface, and the sidewalls, the tool-receiving surface and the outer surface define the cannula fluid channel. The cannula further includes a pair of ribs extending radially between the tool-receiving surface and the outer surface, and the ribs, the tool-receiving surface and the outer surface define the cannula fluid channel. The ribs are positioned along longitudinally extending peripheral edges of the tool-receiving and outer surfaces. The ribs are circumferentially spaced apart from longitudinally extending peripheral edges of the tool-receiving and outer surface. A longitudinal axis of the cannula is angled in the range of 20 to 70 degrees with respect to a longitudinal axis of the handle. The handle includes a handle fluid channel in fluid communication with the cannula fluid channel, and a connector is provided within the fluid passageway configured to connect the handle fluid channel to an external fluid channel. The connector includes a fluid control valve. The handle includes a first end, a second end opposed to the first end and the connector is disposed on the first end. In this embodiment, the first end of the cannula is connected to a second end of the handle, and the handle fluid channel and cannula fluid channel provide a continuous fluid flow pathway between the second end of the cannula and the first end of the handle. The handle includes a first end, a second end opposed to the first end and the connector is disposed on the second end. In this embodiment, the first end of the cannula is connected to a second end of the handle, and the handle fluid channel and cannula fluid channel provide a continuous fluid flow pathway between the second end of the cannula and the second end of the handle.

In some aspects, a device for introducing surgical tools into a body is provided. The device includes a handle, and a cannula fixed to an end the handle. The cannula includes an elongate, hollow, double-walled blade. The blade is shaped such that an inner wall and outer wall are spaced apart. In addition, the inner and outer wall are shaped to form coaxial minor arcs when viewed in a section transverse to a blade longitudinal axis, and the space between the inner and outer wall defines a fluid channel.

Modes for carrying out the present invention are explained below by reference to an embodiment of the present invention shown in the attached drawings. The above-mentioned object, other objects, characteristics and advantages of the present invention will become apparent from the detailed description of the embodiment of the invention presented below in conjunction with the attached drawings.

### BRIEF DESCRIPTION OF THE DRAWINGS

FIG. 1 is a perspective view of an embodiment of the introducing device.

FIG. 2 is a top view of the device of FIG. 1.

FIG. 3 is a side sectional view of the device of FIG. 1.

FIG. 4 is a side view of the second end of the cannula as seen in circle C of FIG. 3.

FIG. 5 is a top view of the second end of the cannula of FIG. 4.

FIG. 6 is a side sectional view of the second end of the cannula as seen along line A-A of FIG. 5.

FIG. 7 is a cross-sectional view of the cannula as seen along line B-B of FIG. 6.

FIG. 8 is another embodiment of the cross-sectional view of the cannula as seen along line B-B of FIG. 6.

FIG. 9 is another embodiment of the cross-sectional view of the cannula as seen along line B-B of FIG. 6.

FIG. 10 is another embodiment of the cross-sectional view of the cannula as seen along line B-B of FIG. 6.

FIG. 11 is a side sectional view of the handle of the device of FIG. 1.

FIG. 12 is a top sectional view of the handle of the device of FIG. 1.

FIG. 13 is a side sectional view of another embodiment of the handle.

FIG. 14 is a top sectional view of the handle of FIG. 13.

### DETAILED DESCRIPTION

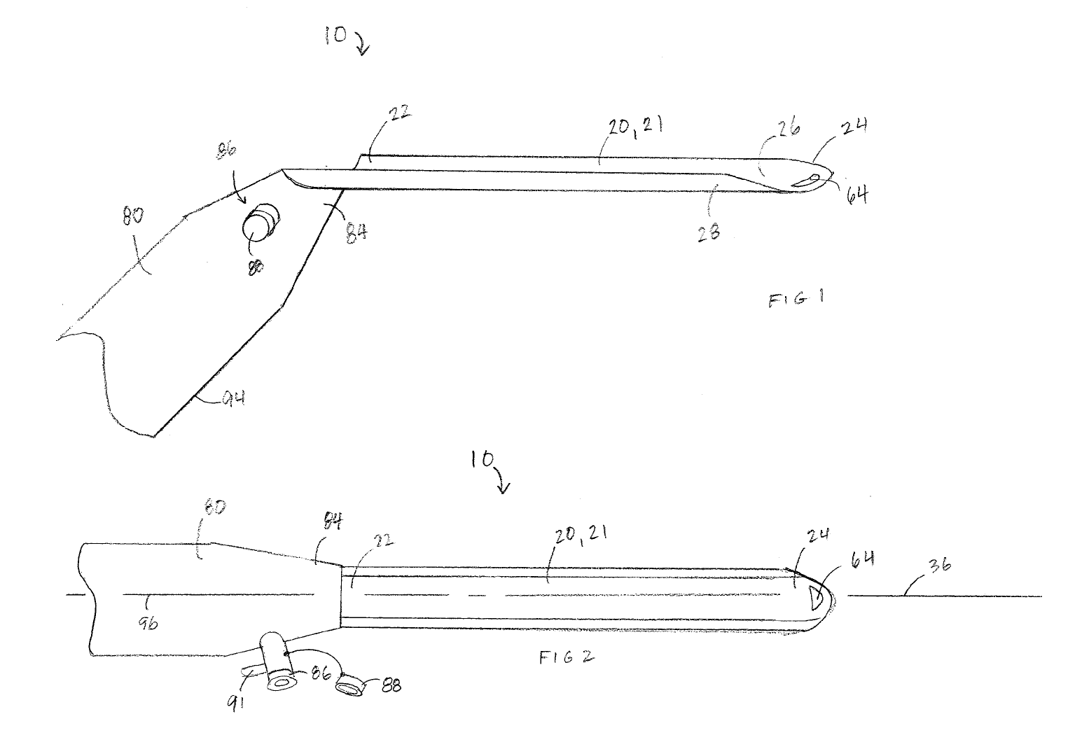

A device 10 for introducing surgical tools into a body will be described with reference to the figures. In the following description, references to orientations such as upper, upward, lower, downward, etc. are made for descriptive purposes with respect to the orientation of the device shown in FIG. 1 rather than to imply any absolute relative orientation.

Referring now to FIG. 1, the device 10 includes a slotted cannula 20 supported on a handle 80. The cannula 20 is an elongate plate 21 having a first end 22 rigidly connected to an end 84 of the handle 80. Elongate plate 21 is curved in the width direction so that the cannula 20 has a cross-sectional shape that is a minor arc opening upward. As a result of this configuration, the cannula 20 includes an upward-facing, concave tool receiving surface 26, and an opposed, downward-facing, convex outer surface 28. In some embodiments, the minor arc has a circumferential arc length in a range between 20 degrees and 180 degrees.

The cannula 20 extends linearly along a longitudinal axis 36, and has a width (e.g. the dimension transverse to the longitudinal direction) that is much smaller than its axial length. In some embodiments, the cannula 20 has a width in a range of 0.5 cm to 2.0 cm, and a corresponding axial length in a range from 3 cm to 13 cm. The particular dimensions of the cannula 20 are determined based on the intended use of the device 10. For example, a cannula 20 to be used in hip arthroscopy may have axial length of about 10 cm, whereas a cannula 20 to be used in knee arthroscopy may have an axial length of about 7 cm, and a cannula 20 to be used in shoulder arthroscopy may have an axial length of about 5 cm. The dimensions disclosed here are presented to provide a relative scale of the cannula 20, and are not intended to be limiting.

The cannula 20 has a second end 24 opposed to the first end 22, the second end 24 corresponding to the end of the device 10 that is inserted into the body. The second end 24 has a rounded shape to ease insertion and to minimize trauma to soft and hard tissues. As seen in FIG. 2, the second end 24 is rounded across the width of the cannula 20. In addition, as seen in FIG. 4, the leading end or tip 25 of the second end 24 is also rounded across the depth of the tip 25. As a further means to ease insertion and minimize injury, the second end 24 is provided with a taper 23 such that the overall depth of the cannula 20 gradually reduces to a minimum depth in the vicinity of the tip 25.

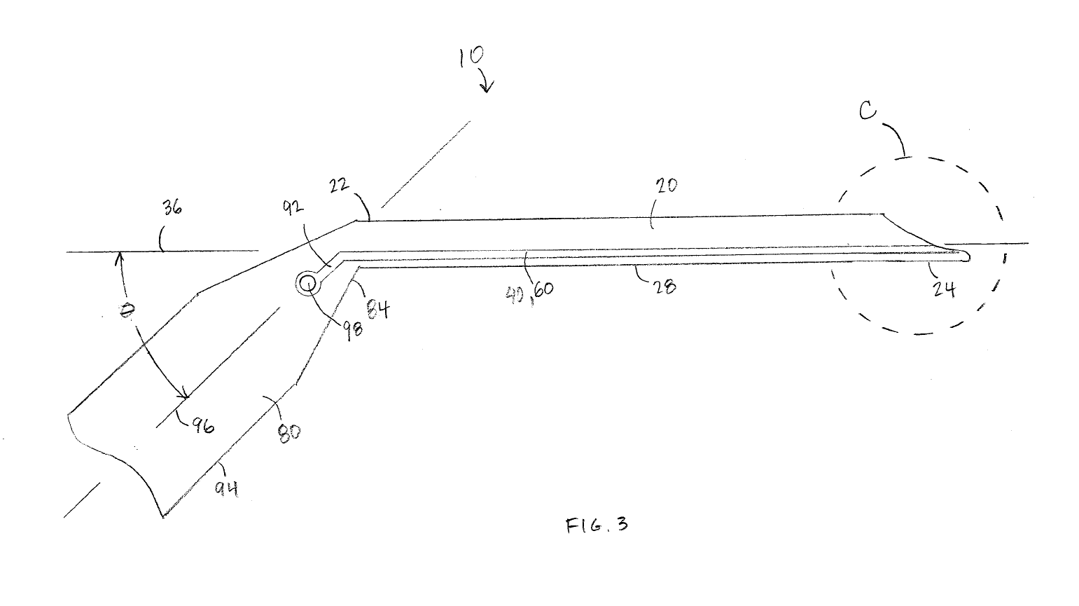

Referring now to FIGS. 3 and 6, the plate 21 of the cannula 20 is a hollow member whereby a vacancy 40 exists between the tool receiving surface 26 and the outer surface 28. The vacancy 40 extends continuously and uniformly (e.g., has uniform dimensions) from the first end 22 to the second end 24 of the cannula 20, and serves as a fluid channel 60 for delivering fluids between the handle 80 and the second end 24 of the cannula 20. Because the fluid channel 60 is disposed between two arcuate surfaces 26, 28, the fluid channel 60 itself is arcuate in cross sectional shape.

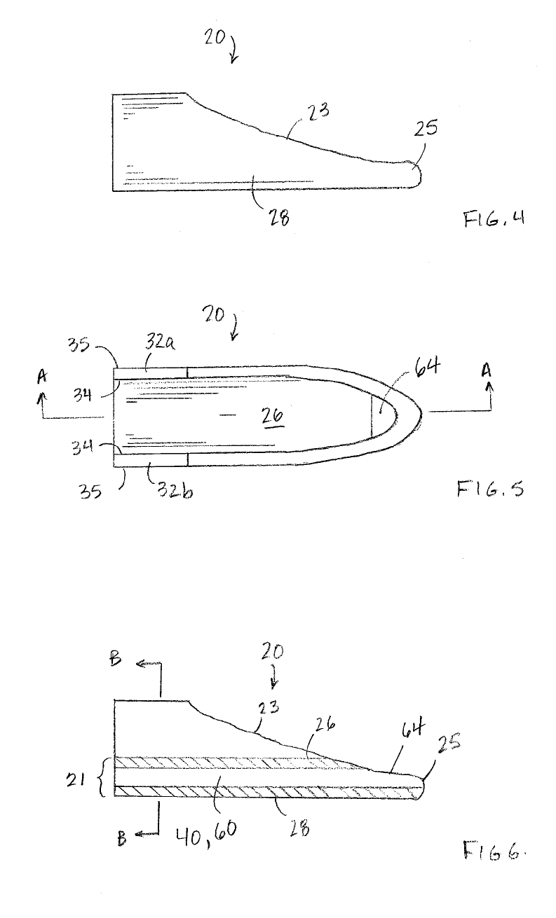

As seen in FIGS. 5 and 6, an opening 64 is provided in the second end 24 of the cannula corresponding to the location at which the fluid channel 60 intersects the taper 23 of the second end 24. As a result, the opening 64 is positioned slightly inward relative to the tip 25. This position is advantageous, since the opening 64 is not obstructed by tissues when the tip 25 is placed abutting a joint surface.

There are many possible configurations of the fluid channel 60. Some of these possibilities will now be described with reference to FIGS. 7-10. In some embodiments, the cannula 20 may include sidewalls 32a, 32b that join respective peripheral side edges 34, 35 of the tool-receiving surface 26 and the outer surface 28. In the embodiment shown in FIG. 7, the sidewalls 32a, 32b, the tool-receiving surface 26 and the outer surface 28 are arranged to define a single fluid channel 60.

Referring to FIGS. 8-10, in some embodiments, the cannula 20 includes at least one rib 62. The rib 62 extends radially between the tool-receiving surface 26 and the outer surface 28. In addition, the rib 62 extends in the axial direction between the first end 22 and second end 24 of the cannula 20. In some embodiments, the rib 62 extends continuously from the first end 22 to the second end 24. The rib 62 is a structural member, providing the cannula 20 with increased rigidity, and also maintaining a uniform spacing between the tool-receiving surface 26 and the outer surface 28 along the axial length of the cannula 20.

As shown in FIG. 8, a single rib 62 may be disposed between the respective sidewalls 32a, 32b (FIG. 8). In the illustrated embodiment, the single rib 62 is disposed midway between the sidewalls 32a, 32b. The vacancy 40 between sidewalls 32a, 32b, the tool-receiving surface 26 and the outer surface 28 is divided by the rib 62, thereby defining two fluid channels 60 within the cannula 20.

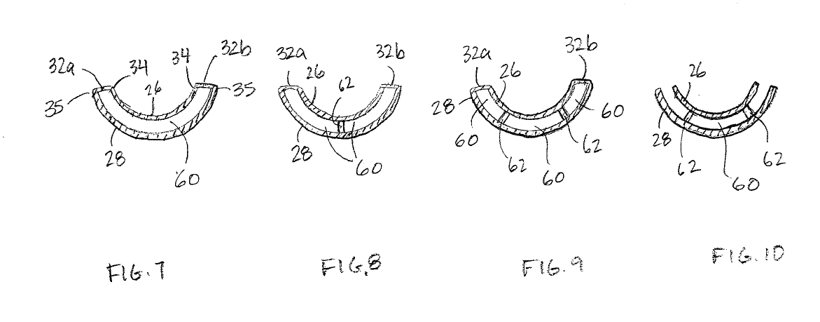

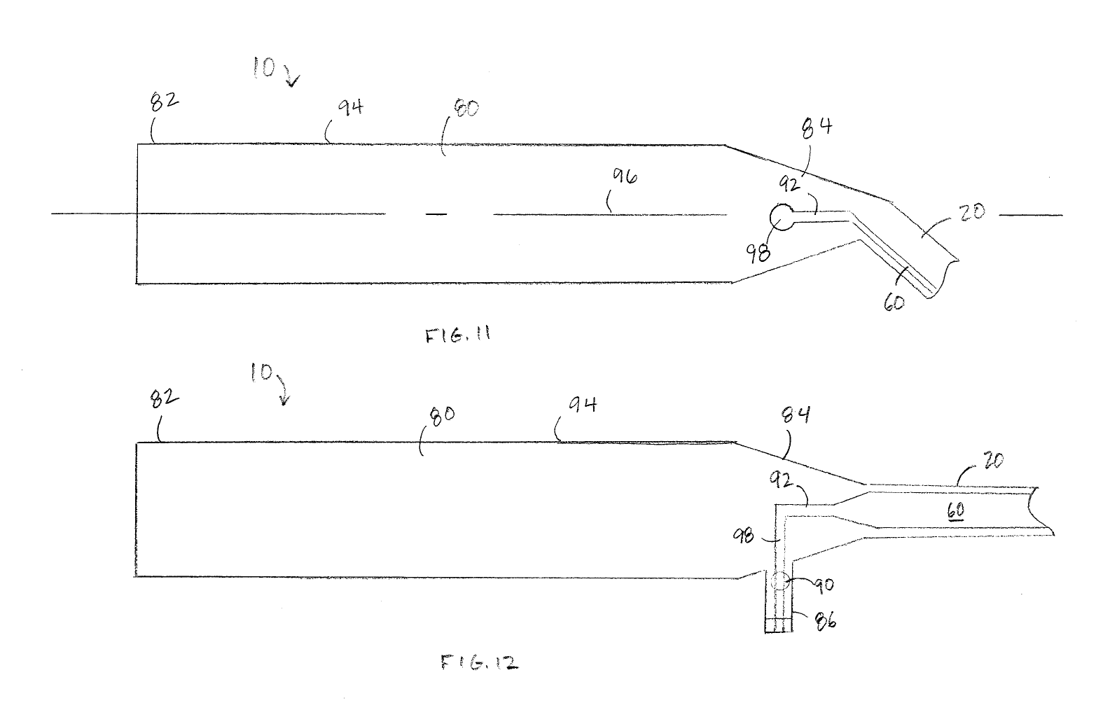

As shown, for example, in FIGS. 9 and 10, more than one rib 62 may be disposed in a spaced-apart manner between the sidewalls 32a, 32b. In some embodiments, the ribs 62 and the respective sidewalls 32a, 32b are equidistantly spaced. For example, in the embodiment illustrated in FIG. 9, two ribs 62 are equidistantly spaced between respective sidewalls 32a, 32b. In this case, the vacancy 40 between sidewalls 32a, 32b, the tool-receiving surface 26 and the outer surface 28 is divided by the two ribs 62 so as to define three fluid channels 60 within the cannula 20.

Referring to FIG. 10, in some approaches, the cannula 20 is not provided with sidewalls 32a, 32b, but does include at least one rib 62; here, in this example, two ribs 62 are provided. In this case, the ribs 62, the tool-receiving surface 26 and the outer surface 28 define a single fluid channel 60.

The one or more fluid channels 60 provided within the cannula 20 each intersect, and are each in fluid communication with, a handle fluid channel 92 provided within the interior of the handle 80 (discussed below).

Referring again to FIG. 3, the cannula 20 extends from the handle 80 at an angle θ. For example, in some embodiments, the longitudinal axis 36 of the cannula 20 is angled in the range of 20 to 70 degrees with respect to a longitudinal axis of the handle 80. In the illustrated embodiment, the longitudinal axis of the cannula is angled at about 45 degrees with respect to a longitudinal axis of the handle 80.

The handle 80 includes an elongate, generally cylindrical body having a first end 82 (for example, the end nearest the user when in use), and a second end 84 opposed to the first end 82. The cannula 20 is rigidly fixed to the second end 84 of the handle 80. Although illustrated somewhat schematically here, it is understood that the outer surface 94 of the handle 80 is shaped and/or is provided with a coating or texture which promotes its grippability and user comfort, and provides other ergonomic benefits during long and/or intense use of the device 10.

The handle 80 further includes a handle fluid channel 92 that communicates with the cannula fluid channel 60. In some embodiments, the handle fluid channel 92 extends within the handle 80 along a longitudinally extending centerline 96.

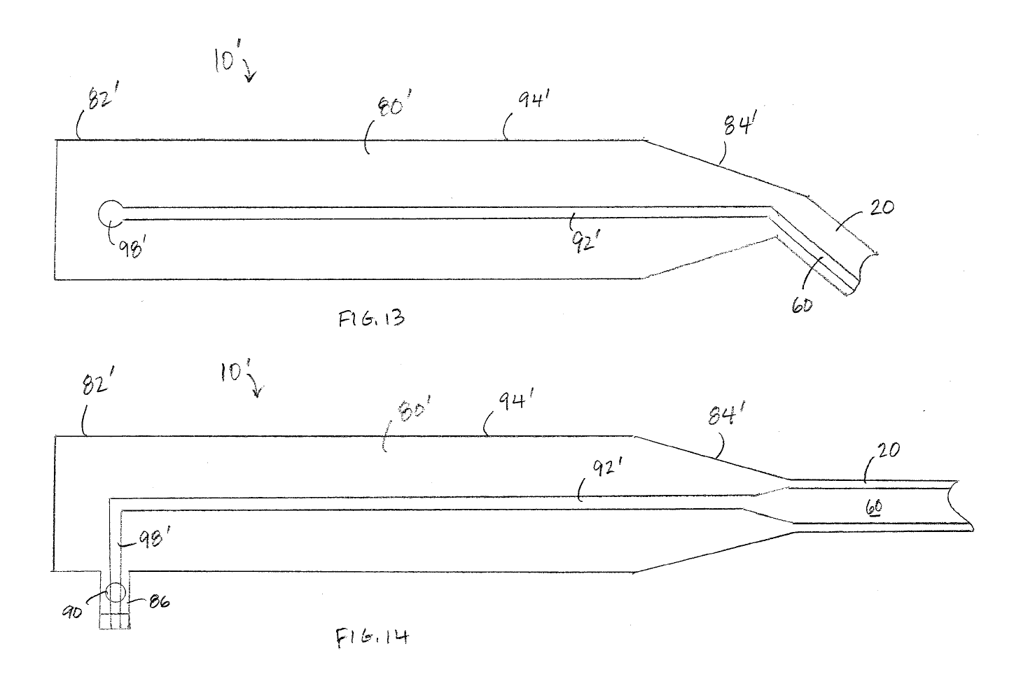

The handle fluid channel 92 extends inward from the second end 84 to a location between the second end 84 and a mid portion of the handle 80. For example, as seen in FIGS. 1-3, 11 and 12, the handle fluid channel 92 extends inward from the second end 84 to a location adjacent to the second end 84. At least one port 98 is provided within the second end 84 of the handle 80 that permits fluid flow between the handle fluid channel 92 and a connector 86 formed on the outer surface 94 of the handle 80. In this embodiment, when the device 10 is held by a user, the connector 86 is disposed between the user's hand and the cannula 20, and thus is easily accessible by some of the user's fingers while a grip is maintained on the handle 80.

In some instances, however, it may be more convenient to have a handle 80 in which the connector 86 is disposed at the first end 82 of the handle. For example, as seen in FIGS. 13 and 14, an alternative handle 80′ is shown in which the handle fluid channel 92′ extends from the second end 84′ to the first end 82′ of the handle 80′. In this embodiment, the connector 86 is provided on the end of the handle 80′ closest to the user. At least one port 98 is provided at the first end 82′ of the handle 80′ that permits fluid flow between the handle fluid channel 92′ and a connector 86 formed on the outer surface 94′ at the first end 82′ of the handle 80′. In this alternative embodiment, when the device 10′ is held by a user, since the connector 86 is disposed on the end of the handle 80′ closest to the user, one of the user's hands grips the handle 80′ between the connector 86 and the cannula 20. In this case, the user would use the other hand to access valves 90 of the connector 86.

The connector 86 may include a removable cap 88. The cap 88 is tethered to the handle to retain the removed cap 88 within easy reach and to prevent accidental loss of the cap 88, especially a loss within a surgical incision. The cap 88 is detachably connected to the connector 86 by conventional means such as a press fit or a threaded engagement.

The connector 86 may include fluid flow control mechanisms, including but not limited to a simple manual valve 90, such as a stopcock, actuated by a stem 91. In addition, the connector 86 may connect to external fluid sources or sinks via conventional means. In some embodiments, the connector 86 includes fittings of the quick connect/disconnect type, such as, but not limited to, a Luer-Lok, which is a registered trademark of Becton Dickinson, Franklin Lakes, N.J., USA.

In some approaches, the device 10 is formed of a metal such as stainless steel. In this approach, the device may be re-used in multiple procedures. In other approaches, the device 10 is formed of an inexpensive, rigid material such as plastic. In this approach, the device 10 may be a disposable device such that it is discarded after a single use.

The device 10 may be sold as part of a kit. For example, in one type of kit, the kit may include multiple versions of the device 10, each version having a unique size and/or handle configuration. In another type of kit, the kit may include one or more versions of the device 10 along with other instruments used for a specific surgical procedure.

A selected illustrative embodiment of the invention is described above in some detail. It should be understood that only structures considered necessary for clarifying the present invention have been described herein. Other conventional structures, and those of ancillary and auxiliary components of the system, are assumed to be known and understood by those skilled in the art. Moreover, while a working example of the present invention has been described above, the present invention is not limited to the working example described above, but various design alterations may be carried out without departing from the present invention as set forth in the claims.

What is claimed is:

1. A device for introducing surgical tools into a body, the device comprising:
a handle; and
a cannula extending from the handle, the cannula including a first end fixed to the handle, and having a shape that is a minor arc in cross section defining
a concave tool-receiving surface, and
a convex outer surface opposed to the tool-receiving surface,
the tool-receiving and outer surfaces extending axially from the handle to a second end of the cannula that is opposed to the first end, and
a cannula fluid channel disposed between the tool-receiving and outer surfaces, the cannula fluid channel extending from the handle to the second end of the cannula, and opening to the cannula exterior at the second end of the cannula.

2. The device of claim 1 wherein the minor arc has a circumferential arc length in a range between 20 degrees and 180 degrees.

3. The device of claim 1, wherein the cannula fluid channel is arcuate in cross sectional shape.

4. The device of claim 1, wherein the cannula further includes opposed sidewalls that join peripheral side edges of the tool-receiving surface and the outer surface, and wherein the sidewalls, the tool-receiving surface and the outer surface define the cannula fluid channel.

5. The device of claim 1, wherein the cannula further includes a pair of ribs extending radially between the tool-receiving surface and the outer surface, and wherein the ribs, the tool-receiving surface and the outer surface define the cannula fluid channel.

6. The device of claim 6 wherein the ribs are positioned along longitudinally extending peripheral edges of the tool-receiving and outer surfaces.

7. The device of claim 6 wherein the ribs are circumferentially spaced apart from longitudinally extending peripheral edges of the tool-receiving and outer surface.

8. The device of claim 1 wherein a longitudinal axis of the cannula is angled in the range of 20 to 70 degrees with respect to a longitudinal axis of the handle.

9. The device of claim 1 wherein the handle further includes
a handle fluid channel in fluid communication with the cannula fluid channel,
a connector provided within the fluid passageway configured to connect the handle fluid channel to an external fluid channel, the connector including a fluid control valve.

10. The device of claim 9 wherein the handle further comprises:
a first end;
a second end opposed to the first end; and
the connector disposed on the first end, and
the first end of the cannula is connected to a second end of the handle, and the handle fluid channel and cannula fluid channel provide a continuous fluid flow pathway between the second end of the cannula and the first end of the handle.

11. The device of claim 9 wherein the handle further comprises:
a first end;
a second end opposed to the first end; and
the connector disposed on the second end, and
the first end of the cannula is connected to a second end of the handle, and the handle fluid channel and cannula fluid channel provide a continuous fluid flow pathway between the second end of the cannula and the second end of the handle.

12. A device for introducing surgical tools into a body, the device comprising:
a handle; and
a cannula fixed to an end the handle, the cannula including an elongate, hollow, double-walled blade, the blade shaped such that
an inner wall and outer wall are spaced apart, and
the inner and outer wall are shaped to form coaxial minor arcs when viewed in a section transverse to a blade longitudinal axis,
the space between the inner and outer wall defining a fluid channel.
